(19) United States
(12) Patent Application Publication
(10) Pub. No.: US 2016/0001000 A1
(43) Pub. Date: Jan. 7, 2016

(54) SYSTEMS AND METHODS USING ESTIMATED GLOMERULAR FILTRATION RATES OF THE KIDNEYS IN THE NON-STEADY STATE

(71) Applicant: Michael Sabry Awadalla, Detroit, MI (US)

(72) Inventor: Michael Sabry Awadalla, Detroit, MI (US)

(21) Appl. No.: 14/740,723

(22) Filed: Jun. 16, 2015

Related U.S. Application Data
(60) Provisional application No. 62/019,518, filed on Jul. 1, 2014.

(51) Int. Cl. A61M 5/172; G06F 19/00

(52) U.S. Cl. CPC A61M 5/1723; G06F 19/3456; A61M 2230/005; A61M 2230/20; A61M 2205/50

(57) ABSTRACT

A system and method of determining the estimated glomerular filtration rate of the kidneys of a patient. The system and method obtains patient medical data, determines constants based on the patient medical data and using exactly one of the MDRD equation or the Cockroft-Gault equation, and determines the estimated glomerular filtration rate based on a relationship of measured creatinine levels and the determined constants. The estimated glomerular filtration rate is used to determine the dose of a medication of a type filtered by the kidneys, determine a temporal correlation of the introduction of a drug into a patient with changes in kidney function, determine the administration rate for dosing intravenous fluids, determine the efficacy of a medical treatment, and determine kidney function after transplantation or injury.

### CROSS-REFERENCE TO RELATED APPLICATIONS

This application claims the benefit of U.S. Provisional Application No. 62/019,518, filed Jul. 1, 2014, which is herein incorporated by reference in its entirety.

### TECHNICAL FIELD

Embodiments of the technology relate, in general, to modeling and estimating the glomerular filtration rate of the kidneys, and in particular to using an estimated glomerular filtration rate to adjust medical treatments and procedures and diagnose kidney function.

### SUMMARY

In an embodiment, a computer-implemented method of determine a dose of medication for a medication that is a type filtered by the kidneys includes accessing an electronic medical record, determining an estimated glomerular filtration rate of the kidneys of the patient, and determining the dose of the medicine for the patient based at least in part on the estimated glomerular filtration rate of the kidneys of the patient. The electronic medical record can include medical data including the age, gender, weight, and ethnicity of a patient, as well as a first measured creatinine level, Cr₁, at a first time, and a second measure creatinine level, Cr₂, at a second time. The estimated glomerular filtration rate is based at least in part on the relationship

$${Cr}_{2} = \frac{A - ( A - B * {Cr}_{1} ) * ( {e}^{B * t} )}{B} ,$$

where t is the interval between the first time and second time, A is a constant determined from the medical data applied to exactly one of the MDRD equation or the Cockcroft-Gault equation, and B is a constant, determined from the medical data applied to exactly one of the MDRD equation or the Cockcroft-Gault equation, that is multiplied by the estimated glomerular filtration rate over the interval. Determining the dose can include determining a standard dose of medication for the patient based on the medical data, determining an adjustment to the standard dose based at least in part on the estimated glomerular filtration rate of the kidney, and the dose is the standard done modified by the adjustment to the standard dose. Determining the dose can also include determining a preferred blood concentration of the medicine for the patient and determining the dose of medicine needed to attain the preferred blood concentration of the medicine in the patient. Determining the dose can also include determining a preferred blood concentration of metabolites of the medicine for the patient and determining the dose of medicine needed to attain the preferred blood concentration of metabolites of the medicine in the patient. Determining the dose can also include determining one or more blood concentrations selected from a preferred blood concentration, a preferred range of blood concentrations, a minimum blood concentration, and a maximum blood concentration of either the medicine or a metabolite of the medicine in the patient, and determining the dose of medicine to attain the one or more blood concentrations. The method can include outputting information about the dose of the medicine and presenting the information on a display.

A non-transitory computer readable medium can have instructions stored thereon that are executed by one or more processors which cause the processors to access an electronic medical record, determine an estimated glomerular filtration rate of the kidneys of the patient, and determine a temporal correlation between the estimated glomerular filtration rate and the introduction of a drug into the patient, and correlate a decrease in the estimated glomerular filtration rate with nephrotoxicity of the drug. The electronic medical record can include medical data including the age, gender, weight, and ethnicity of a patient, as well as a first measured creatinine level, Cr₁, at a first time, and a second measure creatinine level, Cr₂, at a second time. The estimated glomerular filtration rate is based at least in part on the relationship

$${Cr}_{2} = \frac{A - ( A - B * {Cr}_{1} ) * ( {e}^{- B * t } )}{B} ,$$

where t is the interval between the first time and second time, A is a constant determined from the medical data applied to exactly one of the MDRD equation or the Cockcroft-Gault equation, and B is a constant, determined from the medical data applied to exactly one of the MDRD equation or the Cockcroft-Gault equation, that is multiplied by the estimated glomerular filtration rate over the interval. The instructions can further cause the processors to present an indication of the nephrotoxicity of the drug.

A method can include obtaining medical data, determining an estimated glomerular filtration rate of the kidneys of the patient, and determine one or more of an indicia of kidney function, an indicia of the efficacy of a medical treatment, and an administration rate for dosing intravenous fluids based at least in part on the estimated glomerular filtration rate. The medical data can include the age, gender, weight, and ethnicity of a patient, as well as a first measured creatinine level, Cr₁, at a first time, and a second measure creatinine level, Cr₂, at a second time. The estimated glomerular filtration rate is based at least in part on the relationship

$${Cr}_{2} = \frac{A - ( A - B * {Cr}_{1} ) * ( {e}^{- B * t} )}{B} ,$$

where t is the interval between the first time and second time, A is a constant determined from the medical data applied to exactly one of the MDRD equation or the Cockcroft-Gault equation, and B is a constant, determined from the medical data applied to exactly one of the MDRD equation or the Cockcroft-Gault equation, that is multiplied by the estimated glomerular filtration rate over the interval. The method can include correlating the indicia of kidney function with historical kidney transplant data to determine a measure of correlation, and determining a measure of kidney transplant success in the patient based on the measure of correlation. The method can include correlating the indicia of kidney function with an expected range of kidney function to determine a measure of correlation, and quantifying a measure of decreased kidney function in the patient based on the measure of correlation. The method can include diagnosing the patient as having an acute kidney injury based at least in part on the quantified measure of decreased kidney function. The medical treatment can be a medical procedure. The medical procedure can be a surgical medical procedure. The surgical medical procedure can include the removal of an obstruction in a urinary tract. The method can include dosing intravenous fluids into the patient in accordance with the determined administration rate. The method can include determining the estimated glomerular filtration rate of the kidneys in a substantially continuous fashion, determining the administration rate for dosing intravenous fluids into the patient in a substantially continuous fashion, and adjusting the rate of intravenous fluids dosed into the patient in a substantially continuous fashion in accordance with the determined administration rate.

### BRIEF DESCRIPTION OF THE DRAWINGS

The present disclosure will be more readily understood from a detailed description of some example embodiments taken in conjunction with the following figures:

FIG. 1 depicts the physiology of creatinine production by the muscle, distribution in the total body water, filtration by the kidney, and excretion in the urine according to one embodiment.

FIG. 2 depicts a chart of GFR estimates calculated using four different methods for an example patient that has creatinine levels that are changing on a daily basis.

FIG. 3 depicts the flow of information into and out of a medical record and inputs and outputs for software executing on a computing device that determines GFR estimates in accordance to one embodiment.

FIG. 4 depicts an example flow diagram of a method of using dynamic non-steady state glomerular filtration rate estimates to modify treatment of a patient according to one embodiment.

FIG. 5 depicts an example application of using non-steady state GFR estimates to adjust antibiotic dosing for a patient according to one embodiment.

FIG. 6 depicts an example application of using non-steady state GFR estimates to adjust intravenous fluid rate for a dehydrated, post-operative, or septic patient according to one embodiment.

FIG. 7 depicts an example application of using non-steady state GFR estimates to ascertain kidney function and diagnose kidney rejection after transplantation in a patient according to one embodiment.

FIG. 8 depicts an example computing device in accordance with one embodiment.

### DETAILED DESCRIPTION

Described herein are example embodiments of computer-based systems and methods for determining estimated glomerular filtration rates in the kidneys of a patient, and using the estimated glomerular filtration rates to modify treatment options and procedures or to diagnose kidney function.

Various non-limiting embodiments of the present disclosure will now be described to provide an overall understanding of the principles of the structure, function, and use of systems and methods of adjusting medical treatments and procedures and diagnosing kidney function based on estimated glomerular filtration rate of the kidneys of a patient as disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

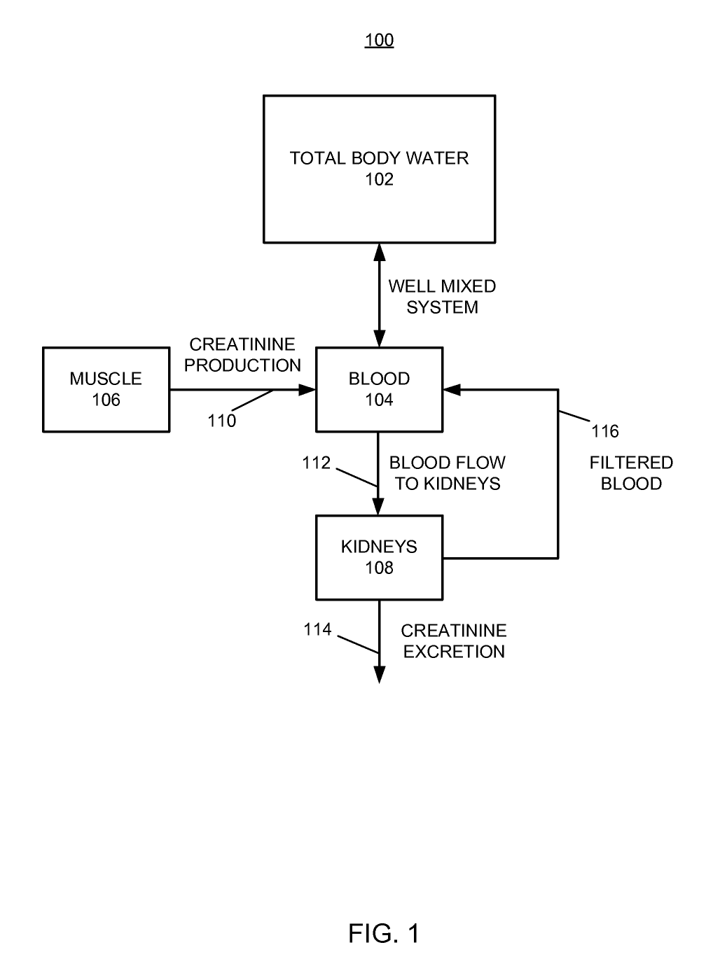

Referring now to FIG. 1, a simplified model 100 is presented of the physiology of creatinine production 110, filtration by the kidneys 108, and excretion 114 through the urine. The model 100 assumes an approximately equal distribution of creatinine over the total body water 102 of the patient which, for a well-mixed system, can be determined by drawing blood 104 and performing laboratory or other well-known measuring techniques. The total body water 102 is assumed to be constant and can be calculated as half of the patient's body weight or by another suitable equation based on patient characteristics such as height and weight. Muscle 106 in the body of the patient produces 110 creatinine which enters the total body water 102 and blood 104 of the patient. Some of the creatinine in the blood flow to the kidneys 112 is filtered by the glomeruli of the kidneys 108 and excreted 114 in the patient's urine thus removing the creatinine from the body of the patient. The filtered blood is then returned 116 from the kidneys 108 back into the bloodstream having had some or all of the creatinine removed.

The level of creatinine in the blood 104 can change not only based on increases in production 110 by the muscles 106 but also based on the filtration 116 and excretion 114 by the kidneys 108. For purposes of this model 100, the production 110 of creatinine is assumed to be produced 110 at approximately a constant rate based on patient characteristics such as age, weight, height, ethnicity, gender, and other suitable patient characteristics. The model 100 also assumes that all of the creatinine that is excreted 114 has been filtered by the glomerui of the kidneys 108 without absorption or additional excretion by the nephron tubules of the kidneys 108. By assuming that the creatinine production 110 by the muscles 106 is constant, and all of the creatinine excreted 114 is filtered by the glomerui of the kidneys 108, the model 100 can correlate changes in creatinine levels in the blood 104 over time with the estimated glomerular filtration rate of the kidneys 108.

Blood 104 from the body of the patient can be sampled and measured at two different points in time. During that time period, which can be a few hours or days, the glomerular filtration rate can be assumed to be approximately constant and the change in creatinine level can be assumed to change continuously from the creatinine level measured in the patient's blood at the beginning of the time period to the creatinine level measured in the patient's blood at the end of the time period.

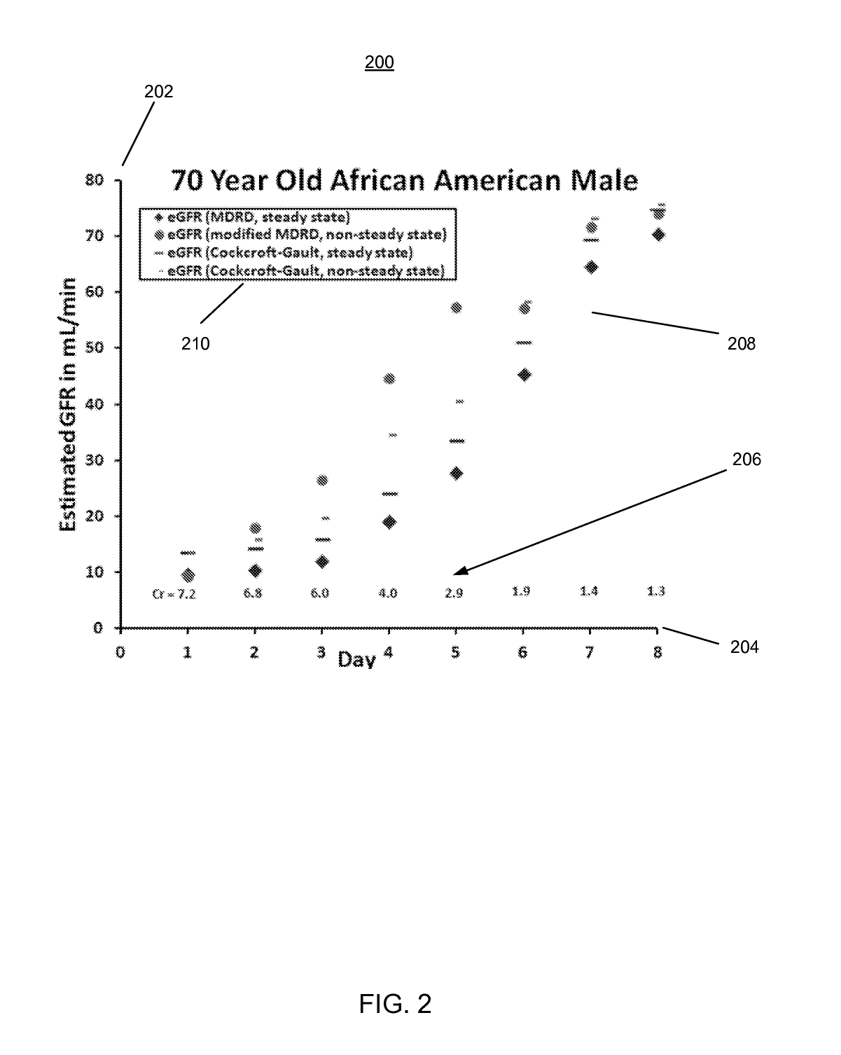

Current methods that are used to estimate the glomerular filtration rate (GFR) in patients include taking a single blood creatinine measurement and apply that measured level to a variable in an equation to determine the patient's GFR. Two such equations, the MDRD equation, or Modification of Diet in Renal Disease equation, and the Cockcroft-Gault equation are limited by their inability to take into account dynamically changing creatinine levels between samples. For example, neither the MDRD equation nor the Cockcroft-Gault equation factor into their equations any rate of change due to the patient's creatinine levels rising or dropping between samples taken. Thus the results of the computed GFRs from the steady state equations are inaccurate in situations when the actual GFR rate of the kidneys is dynamically changing, especially when the GFR rate is changing substantially between samples. An example of this inaccuracy is presented below with regard to FIG. 2, and the accompanying description that illustrates the difference between the steady state equations and the dynamic, non-steady state equations presented below.

Equations for estimating the glomerular filtration rate (GFR) in the non-steady state can be derived as follows. Equation 1 is the Cockcroft-Gault equation in the steady state, or estimated GFR (eGFR_{SS}):

$$eG F {R}_{SS} ( \frac{mL}{min} ) = ( ( 140 - age ) * W * ( 0.85 if female ) ) / ( 72 * {Cr}_{SS} ( \frac{mg}{dL} ) ) \frac{mg * mL}{dL * min} \qquad ( Eq . 1 )$$

where age is the age of the patient, Cr_{SS} is the steady state creatinine concentration, W is the patient's body weight in kg, and a factor of 0.85 is to be applied if the patient is female.

Equation 2 is the estimated creatinine production (Cr_{Prod}), which is the steady state estimated GFR multiplied by the steady state creatinine concentration:

$${Cr}_{Prod} ( \frac{mg}{min} ) = e G F {R}_{SS} ( \frac{mL}{min} ) * {Cr}_{SS} ( \frac{mg}{dL} ) ( \frac{dL}{100 mL} ) . \qquad ( Eq . 2 )$$

Substituting equation 1 into equation 2 yields equation 3:

$${Cr}_{Prod} ( \frac{mg}{min} ) = ( ( 140 - age ) * W * ( 0.85 if female ) ) / ( 7200 ) \frac{mg}{min} \qquad ( Eq . 3 )$$

which is the estimated rate of creatinine production.

The creatinine level of a patient at a future point in time (Cr₊₁(mg/dL)) is the current creatinine level (Crₜ(mg/dL)), plus the additional amount of creatinine produced in the interval, minus the amount of creatinine that is filtered and excreted, as shown in equation 4:

$${Cr}_{t + 1} ( \frac{mg}{dL} ) = {Cr}_{t} ( \frac{mg}{dL} ) + dt ( min ) * ( {Cr}_{Prod} ( \frac{mg}{min} ) ) * ( \frac{1}{{V}_{d ( dL )}} ) - dt ( min ) * ( {Cr}_{t} ( \frac{mg}{dL} ) ) * G F R ( \frac{mL}{min} ) * ( \frac{dL}{100 mL} ) * ( \frac{1}{{V}_{d} ( dL )} ) . \qquad ( Eq . 4 )$$

where GFR is assumed to be constant over a period of time in which the creatinine concentration is not in a steady state, and V_{d} is the volume of distribution of Cr in dL or approximately 500 dL. Substituting equation 3 into equation 4, and substituting the dynamic, non-steady state variable edGFR for GFR yields equation 6:

$${Cr}_{t + 1} = {Cr}_{t} + dt [ ( 140 - age ) * W * ( 0.85 if female ) ] / ( 7200 * {V}_{d} ) - dt ( \frac{{Cr}_{t} * ed G F R}{100 * {V}_{d}} ) \qquad ( Eq . 5 )$$

where edGFR is the dynamic, non-steady state GFR. Rearranging equation 5 yields equation 6:

$${dCr}_{t} = dt ( 140 - age ) * W * ( 0.85 if female ) ] / ( 7200 * {V}_{d} ) - dt ( \frac{{Cr}_{t} * ed G F R}{100 * {V}_{d}} ) \qquad ( Eq . 6 )$$

where dCrₜ is equal to Crₜ₊₁−Crₜ.

For simplicity in integrating the equation, two variables are substituted into equation 6, A=(140−age)*W*(0.85 if female)/(7200*V_{d})) and

$$B = \frac{ed G F R}{100 * {V}_{d}^{′}} ,$$

which yields equation 7:

dCrₜ=dt(A)−dt(B*Crₜ) (Eq. 7)

which can be rearranged to become equation 8:

$$dt = \frac{{dCr}_{t}}{A - B * {Cr}_{t}} \qquad ( Eq . 8 )$$

which when integrated becomes equation 9:

$${\int }_{{Cr}_{1}}^{{Cr}_{2}} \frac{d {Cr}_{t}}{A - B * {Cr}_{t}} = {\int }_{0}^{t} d t \qquad ( Eq . 9 )$$

which resolves to equation 10:

$$- \frac{1}{B} ln ( A - B * {Cr}_{2} ) + \frac{1}{B} ln ( A - B * {Cr}_{1} ) = t . \qquad ( Eq . 10 )$$

Equation 10 can be simplified as shown in equations 11, 12, 13, 14 and 15:

$$\begin{matrix}- \frac{1}{B} ln ( A - B * {Cr}_{2} ) = t - \frac{1}{B} ln ( A - B * {Cr}_{1} ) & ( Eq . 10 ) \\ ln ( A - B * {Cr}_{2} ) = ln ( A - B * {Cr}_{1} ) - B * t & ( Eq . 11 ) \\ A - B * {Cr}_{2} = {e}^{[ l n ( A - B * {Cr}_{1} ) - B * t ]} & ( Eq . 12 ) \\ A - B * {Cr}_{2} = ( A - B * {Cr}_{1} ) * {e}^{( B * t )} & ( Eq . 13 ) \\ {Cr}_{2} = \frac{A - ( A - B * {Cr}_{1} ) * ( {e}^{- B * t} )}{B} . & ( Eq . 14 )\end{matrix}$$

Because B is a function of edGFR, the relationship between edGFR, Cr₁, Cr₂, and t can be mathematically determined, for example by plugging in known values.

For example, if a 25 year old male patient weighing 100 kg with a Vd of 500 dL has his creatinine levels measured over a period of 120 minutes, and his creatinine levels increase from 1.1 to 1.17, then the patient's edGFR using the modified Cockcroft-Gault equation would be equal to 114 mL/min.

Equations for estimating the glomerular filtration rate (GFR) in the non-steady state using the MDRD equation also can be derived. Equation 15 is the MDRD equation in the steady state, or estimated GFR (eGFR_{SS}):

$${eGFR}_{SS} ( \frac{mL}{min} ) = 186.3 * {Cr}_{SS}^{- 1.154} * {Age}^{- 0.203} * ( 0.742 if female ) * ( 1.212 if AA ) \qquad ( Eq . 15 )$$

where Age is the age of the patient, Cr_{SS} is the steady state creatinine concentration, a factor of 0.742 is to be applied if the patient is female, and a factor of 1.212 is to be applied if the patient's ethnicity is African American (abbreviated as AA).

Equation 16 is the estimated creatinine production (Cr_{Prod}), which is the steady state estimated GFR multiplied by the steady state creatinine concentration:

$${Cr}_{Prod} ( \frac{mg}{min} ) = {eGFR}_{SS} ( \frac{mL}{min} ) * {Cr}_{SS} ( \frac{mg}{dL} ) ( \frac{dL}{100 mL} ) . \qquad ( Eq . 16 )$$

Substituting equation 15 into equation 16 yields equation 17:

$${Cr}_{Prod} ( \frac{mg}{min} ) = 186.3 ( \frac{mL}{min} ) * {Cr}_{SS}^{- 1.154} * {Age}^{- 0.203} * ( 0.742 if female ) * ( 1.212 if AA ) * {Cr}_{SS} ( \frac{mg}{dL} ) ( \frac{dL}{100 mL} ) . \qquad ( Eq . 17 )$$

Equation 17 simplifies to equation 18:

$${Cr}_{Prod} ( \frac{mg}{min} ) = 186.3 ( \frac{mL}{min} ) * {Cr}_{SS}^{- 0.154} * {Age}^{- 0.203} * ( 0.742 if female ) * ( 1.212 if AA ) ( \frac{dL}{100 mL} ) \qquad ( Eq . 18 )$$

which is the estimated rate of creatinine production. Because Cr_{SS}^{-0.154} ranges only from 1.20 when

$${Cr}_{SS} = 0.3 \frac{mg}{dL} to .81 when {Cr}_{SS} = 4.0 \frac{mg}{dL} ,$$

it makes it possible to approximate Cr_{SS}^{-0.154} to 1.00 and equation 18 can be further simplified to equation 19:

$${Cr}_{Prod} ( \frac{mg}{min} ) = 1.863 ( \frac{mg}{min} ) * {Age}^{- 0.203} * ( 0.742 if female ) * ( 1.212 if AA ) . \qquad ( Eq . 19 )$$

The creatinine level of a patient at a future point in time

$$( {Cr}_{t + 1} ( \frac{mg}{dL} ) )$$

is the current creatinine level

$$( {Cr}_{t} ( \frac{mg}{dL} ) ) ,$$

plus the additional amount of creatinine produced in the interval, minus the amount of creatinine that is filtered and excreted, as shown in equation 20:

$${Cr}_{t + 1} ( \frac{mg}{dL} ) = {Cr}_{t} ( \frac{mg}{dL} ) + d t ( min ) * ( {Cr}_{Prod} ( \frac{mg}{min} ) ) * ( \frac{1}{{V}_{d ( dL )}} ) - d t ( min ) * ( {Cr}_{t} ( \frac{mg}{dL} ) ) * GFR ( \frac{mL}{min} ) * ( \frac{dL}{100 mL} ) * ( \frac{1}{{V}_{d} ( dL )} ) \qquad ( Eq . 20 )$$

where GFR is assumed to be constant over a period of time in which the creatinine concentration is not in a steady state, and V_{d} is the volume of distribution of Cr in dL or approximately 500 dL. Substituting equation 19 into equation 20 yields equation 21:

$${Cr}_{t + 1} = {Cr}_{t} + d t ( \frac{1.863}{{V}_{d}} * {Age}^{- 0.203} * ( 0.742 if female ) * ( 1.212 if AA ) ) - d t ( \frac{{Cr}_{t} * edGFR}{100 * {V}_{d}} ) \qquad ( Eq . 21 )$$

where edGFR is the dynamic, non-steady state GFR. Rearranging equation 21 yields equation 22:

$$d {Cr}_{t} = d t ( \frac{1.863}{{V}_{d}} * {Age}^{- 0.203} * ( 0.742 if female ) * ( 1.212 if AA ) ) - d t ( \frac{{Cr}_{t} * edGFR}{100 * {V}_{d}} ) \qquad ( Eq . 22 )$$

where dCrₜ is equal to Crₜ₊₁−Crₜ.

For simplicity in integrating the equation, two variables are substituted into equation 21,

$$A = \frac{1.863}{{V}_{d}} * {Age}^{- 0.203} * ( 0.742 if female ) * ( 1.212 if AA ) and B = \frac{edGFR}{100 * {V}_{d}} ,$$

to yield equation 7 as shown above for the Cockcroft-Gault equation:

dCrₜ=dt(A)−dt(B*Crₜ). (Eq. 7)

Equation 7 can be solved using the steps associated with equations 8 through 13 found above for the Cockcroft-Gault equation, which ultimately resolves to equation 14:

$${Cr}_{2} = \frac{A - ( A - B * {Cr}_{1} ) * ( {e}^{- B * t} )}{B} . \qquad ( Eq . 14 )$$

Because B is a function of edGFR, the relationship between edGFR, Cr₁, Cr₂, and t can be mathematically determined, for example by plugging in known values.

For example, if a 25 year-old Caucasian male patient with a Vd of 500 dL has his creatinine levels measured over a period of 120 minutes, and his creatinine levels increase from 1.1 to 1.17, then the patient's edGFR using the modified MDRD equation would be equal to 59.6 mL/min.

Therefore, as described in the above equations and detailed description, a differential equation of the form dCrₜ=dt(A)−dt(B*Crₜ) can constructed based on the above assumptions where “A” is a constant and “B” is a constant multiplied by the glomerular filtration rate over the time period. The equation is integrated from the time (t₁) when a first blood creatinine level (Cr₁) is measured to the time (t2) when a second or subsequent creatinine level (Cr₂) is measured. The time interval (t) is expressed in minutes and the final equation in its simplified form as expressed in equation 14 above. By determining values for A and B that are calculated from a patient's age, height, weight, gender, and/or ethnicity, and plugging those values into equation 14, the above equations can be used to estimate the non-steady state, dynamic glomerular filtration rate of a patient's kidneys with greater accuracy. While the examples described herein use a mathematical solution to demonstrate solving the differential equation dCrₜ=dt(A)−dt(B*Crₜ) in specific scenarios, an analytical solution can also be used for a given A, B, t₁, t₂, Cr₁, and Cr₂.

Referring now to FIG. 2, a chart 200 of a series of steady-state and non-steady state estimates of the glomerular filtration rate (GFR rate) of an example 70 year-old African American patient are presented. The chart 200 illustrates how a patient is likely to benefit from calculation of an estimated GFR rate that corrects for dynamically changing creatinine levels between samples. The chart 200 includes two axis, a vertical axis for the estimated GFR 202, and a horizontal axis for the timeline, in this case days 204 that samples of blood were taken. The measured creatinine levels 206 in the blood of patient are illustrated as the numbers Cr 7.2, 6.8, 6.0, . . . , 1.3 and show that the level of creatinine, Cr, in the blood of the patient was decreasing, which is generally a sign of improving kidney function as the kidney is able to filter the creatinine out of the blood.

However, although the GFR estimates 208 generally show that the patient's kidney function was generally improving, the steady-state estimates of the MDRD and Cockcroft-Gault equations fail to show the true measure of the patient's improvement. Referring to the legend 210 of the chart 200 and the GFR estimates 208, the diamonds for the steady-state MDRD estimates appear to show that the patient did not really start to improve dramatically until about day four, and only dramatically recovered between days 5 and 7 when the slope of the line between the diamonds is greatest. However, as shown by the spheres representing the non-steady state, dynamic modified MDRD estimates described in this specification, the patient's glomerular filtration rate was improving substantially right from day 1, recovered the fastest between days 3 and 5, and actually may have had a slowdown in recovery between days 5 and 6. Similarly, the non-steady state, dynamic modified Cockcroft-Gault estimates, illustrated as short horizontal bars, shows that there was better improvement in the patient's glomerular filtration rate in the first several days than was shown in the steady-state Cockcroft-Gault estimates which are illustrated as longer horizontal bars.

If the improvement was due to a procedure, such as removing an obstruction in the urinary outflow tract, or the introduction of a drug to improve kidney function, a physician using the steady-state MDRD or Cockcroft-Gault estimates may not have recognized that the treatment was as effective as the non-steady state modified MDRD or Cockcroft-Gault estimates both clearly show. Further, the apparent lack of immediate improvement in the patient could trigger a less experienced physician to change the treatment whereas the non-steady state modified MDRD or Cockcroft-Gault estimates show that the treatment was effective from day 1. Further, a physician seeing the steady-state MDRD estimate on day 6 would interpret the GFR estimate 208 as the best improvement seen to date, whereas the non-steady state modified MDRD shows that there was actually an inflection point where the effectiveness of the recovery of kidney function appears to have decreased slightly. Without the non-steady state modified MDRD, it might be difficult for a physician to detect an early warning sign of a potential problem, especially given the fact that the GFR estimate 208 for day 6 also shows an improvement in the creatinine level over day 5. Had there actually been a worsening of kidney function, it could go unnoticed for another day or so without the non-steady state modified MDRD.

The significance of using these methods in certain clinical scenarios to estimate the GFR in the non-steady state can be seen by observing that on days 2 through 5, the estimated GFR by the modified MDRD equation in the non-steady state is twice what the steady-state MDRD equation predicts. Therefore, FIG. 2 illustrates an example scenario of a patient who is likely to benefit from calculation of the estimated GFR using an equation that corrects for the changing creatinine level over time, such as the non-steady state MDRD and Cockcroft-Gault equations and methods described above.

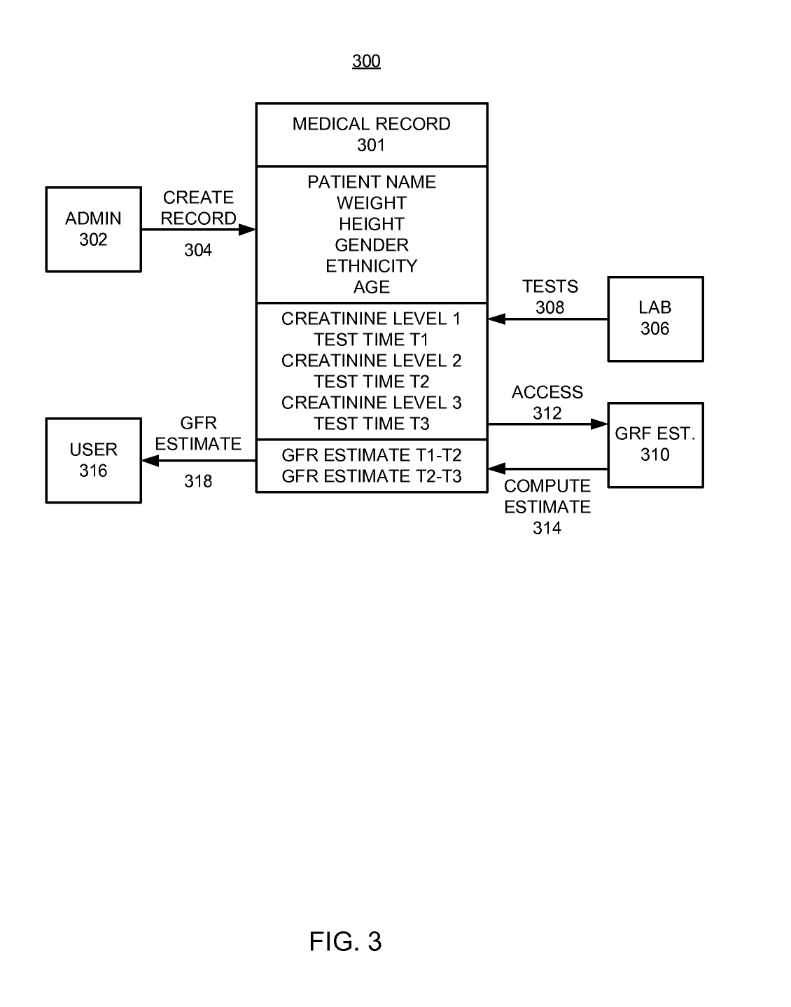

Referring now to FIG. 3, the flow of information into and out of a software program for determining non-steady state GFR estimates is presented. The flow of information is presented using an example medical records system 300 for ease of explanation only, and is not intended to limit the invention thereby. The electronic medical record 301 can reside in a hospital's main medical record system or another system used to store patient information such as a mobile device application where a health care provider enters patient information and laboratory data. The private patient information stored in the medical records can be secured, for example by using passwords, encryption, and authentication methods known in the art or yet to be developed.

An administrative assistant 302, doctor, or other person creates 304 a medical record 301 associated with the patient in the medical records system 300. Sample patient information that can be entered can include the patient's name, weight, height, gender, ethnicity, age, and other information including address, family members, insurance, and so forth.

The patient can have blood drawn by a lab 306 and the test results 308 can be entered into the patient's medical record 301. For example, after the first test at time t1, the first creatinine level and test time can be entered into the patient's medical record 301. The second time the patient has a test performed at time t2, the second creatinine level and test time can be entered into the patient's medical record 301. The third time the patient has a test performed at time t3, the third creatinine level and test time can be entered into the patient's medical record 301, and so forth.

The software program for determining non-steady state GFR estimates 310 can access the patient's medical record 301 and retrieve the data for the creatinine level at time t1, and the creatinine level at time t2. The software program represents software using the calculation of the estimated glomerular filtration rate in the non-steady state. The software program for determining non-steady state GFR estimates 310 can compute the estimate 314 for the GFR from time t1 to time t2, which can be entered into the patient's medical record 301. The software program for determining non-steady state GFR estimates 310 can do the same for the creatinine levels for time t2 and t3 and enter the GFR estimate for time t2 to time t3 into the patient's medical record 301. This process is described for two time intervals (three separate creatinine measurements) by way of example only. In actual practice, there could be only one time interval or many more time intervals.

A user 316 can retrieve the GFR estimates 318 and use them for modifying treatment options. The user 316 could represent a physician, nurse, pharmacist, researcher, an electronic medical record system, another software program, a mobile device application, or any other suitable user, entity, or system.

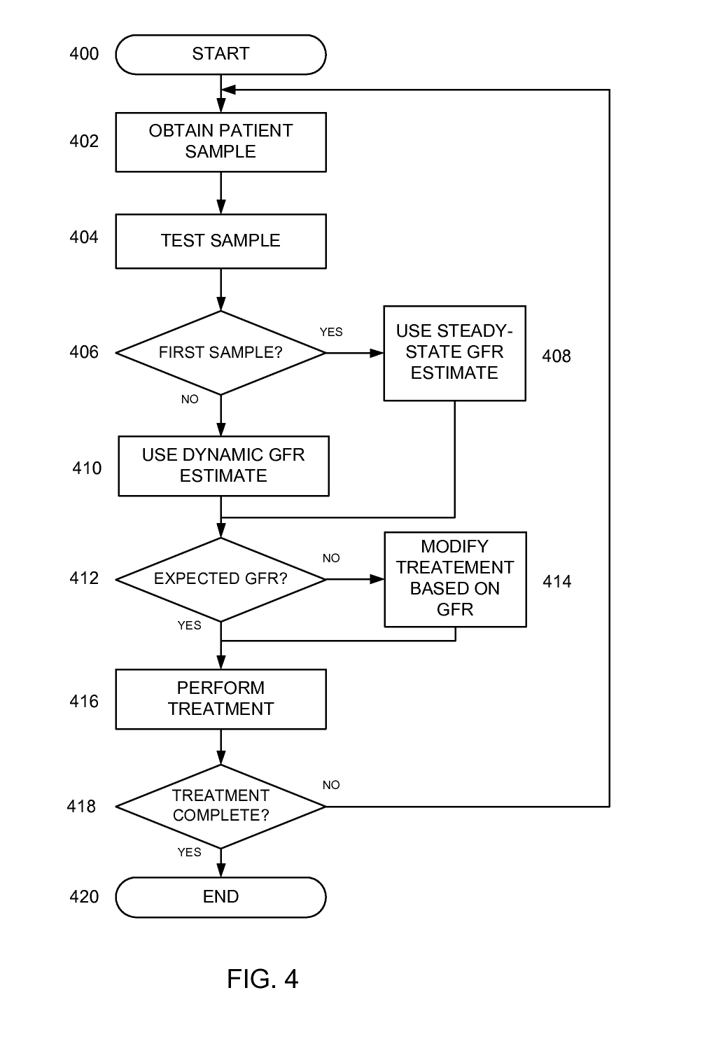

Referring now to FIG. 4, an example flow diagram of a method of using non-steady state dynamic GFR estimates to treat a patient is presented. Processing starts at start block 400 and continues to process block 402.

In process block 402, a sample is obtained from a patient. For example, blood can be drawn by a phlebotomist at a lab. The blood sample can represent serum or plasma. Other suitable fluids or samples from the patient can also be taken. For example, if excreted creatinine is to be measured, then a urine sample can be obtained. Processing continues to process block 404.

In process block 404, the sample is tested. For example, a laboratory can test the sample to determine the amount of creatinine present in the sample. The laboratory can enter the information into a medical system as part of the patient's medical record for example. The information can be stored or entered into the system using any suitable data format, for example an automatic entry of the creatinine level into a computer system, or by having the data input by the clinician into a web page of a medical records system. Processing continues to decision block 406.

In decision block 406, if this is the first sample that has been taken and measured in process blocks 402 and 404 respectively, then processing continues to process block 408, otherwise processing continues to process block 410.

In process block 408, the glomerular filtration rate is estimated using steady state GRF estimation procedure. For example, the steady-state MDRD or Cockcroft-Gault equations can be used to determine the GFR estimate. The GFR estimate can be stored, for example in a medical record for the patient in the medical records system. The steady-state MDRD or Cockcroft-Gault equations are used for the first sample because there is only a single sample that has been taken. Once there are more samples that have been taken at intervals, the dynamic, non-steady state modified equations can be used as described above and in process block 410. Processing continues to decision block 412.

In process block 410, the glomerular filtration rate is estimated using the dynamic, non-steady state GRF estimation procedure described above. The dynamic, non-steady state GFR estimate can be determined using the modified MDRD or Cockcroft-Gault equations described above. For example, the creatinine level for a previous sample, and the creatinine level of the current sample from process blocks 402 and 404, can be used to determine the dynamic, non-steady state GRF estimate. The GFR estimate can be stored, for example in a medical record for the patient in the medical records system. Processing continues to decision block 412.

In decision block 412, if the glomerular filtration rate of the GFR estimate is within a desired range then processing continues to process block 416. Otherwise, processing continues with process block 414.

In process block 414, the treatment regimen for the patient can be modified based on the glomerular filtration rate. For example, if the GFR estimate is too low, then treatment options can be modified to increase the glomerular filtration rate. For example, if IV or intravenous fluids are being given, and the glomerular filtration rate is too low, then the treatment regimen can be changed. For example, the treatment regimen can be modified to increase fluids or give medicine to increase the glomerular filtration rate in the kidneys. Processing continues to process block 416.

In process block 416, the treatment can be performed. The treatment can be based on the needs of the patient. For example, if the procedure is outpatient monitoring, then the patient can continue on a health or diet regimen as directed by their physician. If the patient is undergoing dialysis, then dialysis can be considered. If the patient is being given fluids as part of a resuscitative effort or post-op, then fluids can be given. The treatment can be modified as discussed in process block 414. Processing continues to decision block 418.

In decision block 418, if ongoing treatment of the patient is desired, the processing continues at some point in time back at process block 402. If treatment is complete, then processing ends at end block 820.

Generally, the operations described in process blocks and decision blocks 400 through 420 can be performed in any order, as would be understood by one of ordinary skill in the art. For example, the treatment performed in process block 416 can be continuously modified based on the GFR as discussed in process block 414 even if the glomerular filtration rate is in an expected range. Processing does not have to end at end block 420, but can continue in a loop starting with any suitable process block or decision block.

The systems and methods described herein can provide diagnostic criteria for acute kidney injury to be based on changes in estimated GFR rather than changes in serum creatinine which is the current standard. Since GFR is an indication of kidney perfusion, the systems and methods can allow for titration of intravenous fluid administration and pressor medications to achieve adequate perfusion of an end organ such as the kidney by directing therapy toward the goal of a specific GFR or change in GFR over time.

The systems and methods described herein are applicable to different kinds of patients and medical needs. A patient can be an outpatient, an inpatient, an ICU patient, a patient undergoing a surgery, a kidney transplant patient, a trauma victim, a dialysis patient, and so forth. The estimated GFR can be used to adjust dosing of medications and intravenous fluids used in a patient's care, provide medical diagnosis, and determine the efficacy of procedures.

For example, in dialysis patients the described systems and methods can be used to determine residual kidney function. Residual kidney function could be calculated by the above described methods provided that the time points at which the patient's creatinine levels are measured do not span the course of a dialysis treatment. For instance, two creatinine measurements could be used if the first measurement was immediately after a dialysis treatment and the second immediately before the next dialysis treatment.

In dialysis patients, the systems and methods described herein can be used to determine the effectiveness of a dialysis treatment. Because creatinine is filtered out of the blood with dialysis, a blood creatinine level drops over the course of a dialysis session. The effectiveness of a dialysis session can be calculated in the same way as an estimated GFR is calculated. The creatinine level before and after dialysis would be measured and the estimated GFR during the dialysis session would correlate with the rate at which the dialysis removed creatinine and other similarly sized molecules from the blood. The estimated GFR over the dialysis session multiplied by the time in minutes of the dialysis session would provide a measure of the creatinine clearance of the dialysis session.

The systems and methods can enable the monitoring of small changes of a patient's GFR over short periods of time. Such monitoring can detect drug nephrotoxicity which would manifest as a recognizable drop in GFR within a specific time period after a particular drug had been administered. This would allow early cessation of the nephrotoxic drug and prevent continued kidney damage. For example, a non-transitory computer readable medium having instructions stored on it could be executed by a processor that would cause the processors to access an electronic medical record having patient medical data. The medical data could be used to determine an estimated glomerular filtration rate of the kidneys of the patient using the equation

$${Cr}_{2} = \frac{A - ( A - B * {Cr}_{1} ) * ( {e}^{- B * t} )}{B}$$

as described above. A temporal correlation can be determined between the estimated glomerular filtration rate and the time at which the drug was introduced into the patient and would have started to take effect. If the estimated glomerular filtration rate decreased, then the nephrotoxicity of the drug can be correlated with the decrease in the estimated glomerular filtration rate and the use of the drug with the patient can be discontinued.

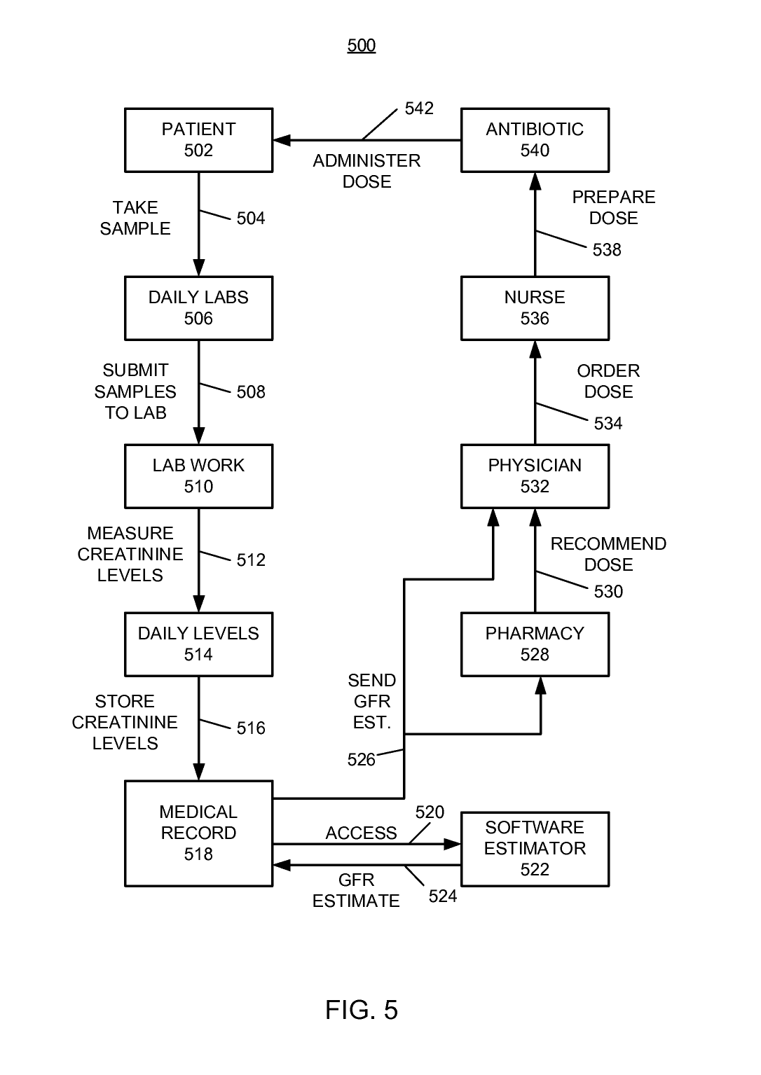

Referring now to FIGS. 5, 6, and 7, several specific applications for using modified non-steady state GFR estimates are presented. FIG. 5 depicts a detailed view of how the dosing levels of antibiotics and other medications for a patient can be determined or adjusted according the glomerular filtration rate of a patient's kidneys. A system 500 for monitoring creatinine levels and adjusting the dose of antibiotic based on the glomerular filtration rate is presented. A patient 502 can be a hospitalized patient. Many hospitalized patients have their body fluids, such as blood, sampled 504 as part of daily labs 506. For example renal function panels or basic metabolic panels are often measured daily (both panels include creatinine levels). These results could be used by physicians 532 to adjust medication doses on a daily basis. In general this system 500 would relate to determining two blood creatinine levels that are measured on the morning of consecutive days, as is already common practice in inpatient hospital settings.

The samples are submitted 508 to a lab 510, normally in the hospital where the patient 502 is hospitalized. The lab 510 measures 512 the creatinine levels 514 and the creatinine levels 514 are stored 516 in the patient's medical record 518. The glomerular filtration rate can be measured by software 522, for example as described above with reference to FIG. 3. The software 522 accesses 520 the patient's medical record 518, retrieves one or more daily creatinine levels 514, and determines the GFR estimate 524 which is stored in the patient's medical record 518. The software 522 can run within another software program (such as a hospital's electronic medical record system) or an independent program such as a mobile device application.

The GFR estimate 524 can be sent 526 to the pharmacy 528 or the physician 532 or any other suitable person, system, or computing device. For example, in a configuration, the nurse 536 or an antibiotic drug delivery system (not shown) could receive the GFR estimate. For example in such a configuration, the nurse or antibiotic drug delivery system could be used to make the dosing adjustments with or without physician review and approval. The GFR estimate 524 can also be displayed on a monitor, printed on a label, or otherwise presented. The pharmacy 528 can use the GFR estimate 524 to send the next recommended dose 530 of antibiotic 540 for the patient 502 to the physician 532. The physician 532 can use the GFR estimate 524 and recommended does 530 and write an order 534 for the next dose of antibiotic 540 to be given to the patient 502. A nurse 536 can receive the order 534, prepare 538 the dose of antibiotic 540, and administer 542 the dose of antibiotic 540 to the patient 502.

In some embodiments, this system 500 could be used to monitor a patient's kidney function every time they have a creatinine level measured. This would allow accurate dosing of medications that could be calculated automatically by another software program and updated daily or hourly. Specifically, the dose and/or duration of time between doses could be updated every time a patient has a creatinine level measured.

In various embodiments, the GFR estimate can be used to determine different drug regimens and prescriptions for the patient. For example, in one embodiment, a standard dose of medicine can be determined for the patient using the various medical data available for the patient. The standard dose can then be increased, decreased, or otherwise modified based on the GFR estimate. In another embodiment, a preferred concentration of the medicine in the blood of the patient may be desirable. Using the GFR estimate, and assuming that the medicine will be filtered by the kidneys at approximately the same rate as the creatinine is filtered by the kidneys, the dose of medicine necessary to attain that preferred concentration can be determined. In another embodiment, the medicine may become effective once it is turned into a metabolite in the body of the patient. In this case, the dose of medicine needed to attain a preferred blood concentration of the metabolite in the blood can be determined. In another embodiment, there may be a range of acceptable blood concentrations for either the medicine or a metabolite in the blood. The GFR estimate can be used to determine the dose of medicine needed to maintain the blood concentration in the acceptable range. Similarly, by knowing the glomerular filtration rate, a schedule of doses of medicines and a timetable for taking the medicines can be determined to attain target blood concentrations of the medicine or a metabolite of the medicine. Similarly, the dose can be determined that would result in preferred blood concentrations, preferred ranges of blood concentrations, minimum blood concentrations, and maximum blood concentrations of either the medicine, a metabolite of the medicine, or both.

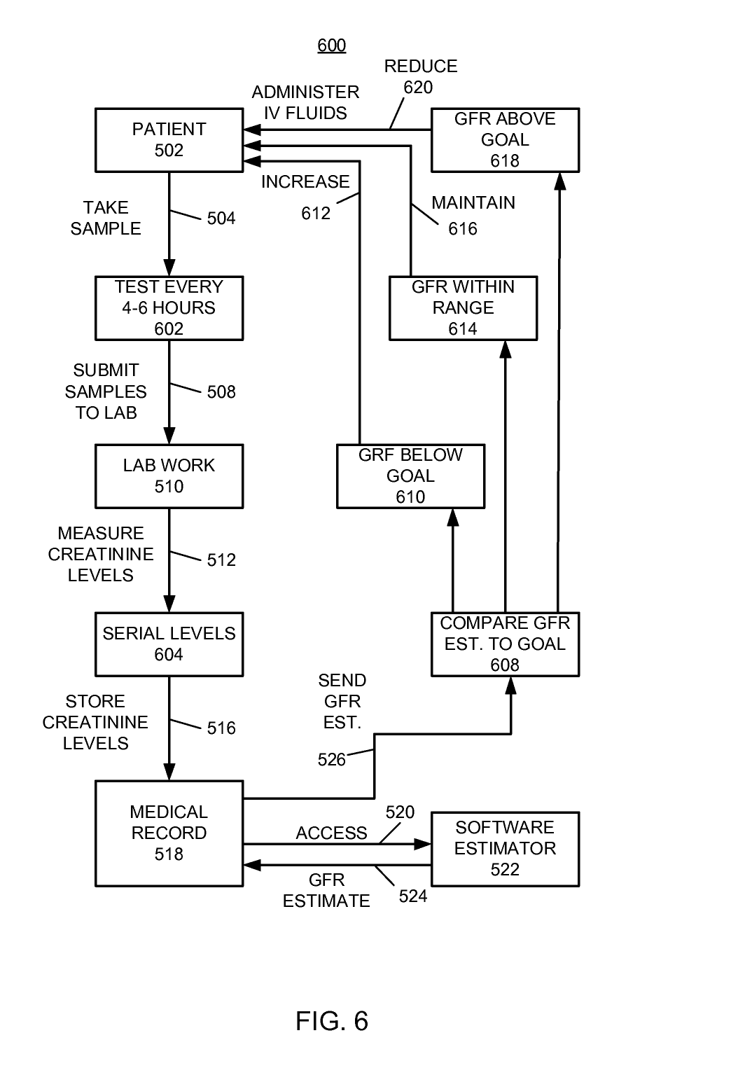

Referring now to FIG. 6, a detailed view of how a system 600 can be used to titrate intravenous fluid administration rates. In this example, the patient 502 may be admitted to an intensive care unit and intravenous fluid may need to be administered, for example to resuscitate a dehydrated patient, or provide fluids to a patent 502 in a post-operative condition, or to increase blood pressure and end organ perfusion. In this system, the blood may be drawn 602 every few hours. The samples would be sent to the lab 510 as described for FIG. 5, and serial creatinine levels 604 can be determined and entered into the medical record 518 of the patient. The software 522 can determine GFR estimates 524.

The GFR estimate 524 based on the serial creatinine levels 604 can be sent 526 to a second computer algorithm that is configured to compare 608 the GFR estimate 524. The second computer algorithm can compare 608 the GFR estimate 524 to a target range of glomerular filtration rates, one or more threshold rates, or other suitable goal or set of goals. For example, in different configurations, the goal estimated GFR can be a fixed value such as 60 milliliters per minute, or a changing value such as 45 milliliters per minutes on the first day, and then 60 milliliters per minute on a subsequent day. Other suitable goals as would be understood in the art could be used. If the GFR is determined to be below 610 the goal rate, then an increased amount of IV fluids 612 can be administered to the patient. If the GFR is determined to be within an acceptable range 614, then the IV fluids can be maintained 616 at the current rate. If the GFR is determined to be above 618 the goal rate, then the IV fluids can be reduced 620, and could be ceased altogether. In a configuration, the IV fluids could also be maintained even when the GFR is determined to be above 618 the goal rate.

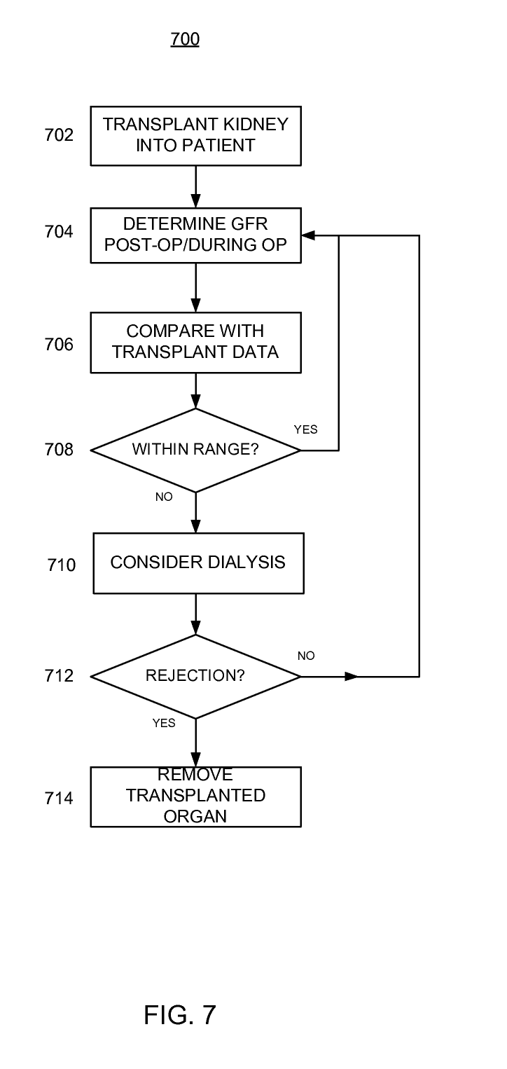

Referring now also to FIG. 7, for kidney transplant recipients, being able to sense changes in the glomerular filtration rate can be helpful as an early indicator of transplant rejection. Currently transplanted kidneys are monitored by ultrasound to evaluate for blood flow through the transplanted kidney. The system 700 presented can allow for evaluation of kidney function at a biochemical level. A kidney that is functioning well would be expected to clear creatinine at a rate similar to that of other successfully transplanted kidneys. A kidney that is being rejected by the recipient's body would be expected to clear creatinine at a lower rate and have a lower estimated GFR using the modified non-steady state equations presented herein. Using the non-steady state equations to estimate GFR can permit early recognition of kidney rejection in the first few hours or days after transplant. Early recognition of kidney rejection can help in preparing a patient for needed dialysis or an operation to have the transplanted kidney surgically removed or exchanged for a new kidney. It is possible that this system could even be used to measure the transplanted kidney's function intraoperatively which would allow kidneys to be tested and exchanged based on their function biochemically with a given recipient.

FIG. 7 presents example operations of a method 700 for determining kidney function for transplant recipients. In step 702, a kidney is transplanted into the patient. In step 704, creatinine levels in the transplant recipient are measured and the patient's glomerular filtration rate is determined using one or more of the dynamic, non-steady state methods described above for FIG. 3. For example, the modified MDRD or Cockcroft-Gault equations can be used to estimate GFR. If the measuring is occurring during the operation, or immediately post-op, then the measuring can be performed in short intervals, for example several minutes apart. As the transplant patient recovers, the testing intervals can be longer, for example every hour or several hours apart. In step 706, The GFR after each test can be compared with the GFR and creatinine levels of other transplant patients to determine if the patient's glomerular filtration rate is within the expected range seen during other successful kidney transplants or if the patient's glomerular filtration rate is not within the anticipated range. This could indicate that the transplanted kidney is not functioning as well as expected, or that the kidney is being rejected by the patient's body. In step 708, if the estimated GFR is within the expected range, then the patient can continue to be periodically tested in step 704. If the estimated GRF is not within the expected range based on other kidney transplants, then in step 710, dialysis can be considered to replace the missing filtration. In step 712, if there is no rejection of the kidney, then the patient can continue to be periodically tested in step 704. If the kidney is being rejected, then in step 714 the transplanted organ can be removed. If another kidney is available for transplantation, then the process can begin again at step 702 and the new kidney can be transplanted into the patient.

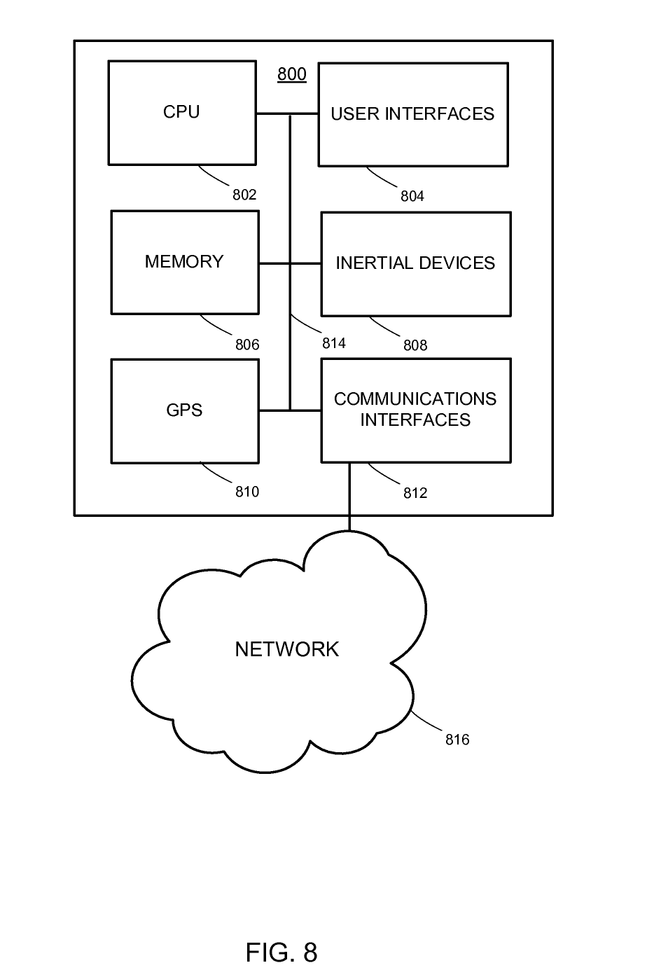

Referring now to FIG. 8, an example computing device 800 for executing the non-steady state GFR estimate software is presented. The non-steady state GFR estimate software can run on any suitable computing system. The processes described herein can be performed on or between one or more computing devices 800. A computing device 800 can be a server, a computing device that is integrated with other systems or subsystems, a mobile computing device, a cloud-based computing capability, a dedicated server, a personal computer, multiple computers, a collection of networked computers, a cloud-based computer system, a web-based computer system, and so forth. One or multiple processing units, such as central processing units and/or graphics processing units, can perform instructions stored in memory to execute the processes described herein.

The computing device 800 can be any suitable computing device as would be understood in the art, including without limitation, a custom chip, an embedded processing device, a tablet computing device, a personal data assistant (PDA), a desktop, a laptop, a microcomputer, a minicomputer, a server, a mainframe, or any other suitable programmable device. In various embodiments disclosed herein, a single component can be replaced by multiple components and multiple components can be replaced by a single component to perform a given function or functions. Except where such substitution would not be operative, such substitution is within the intended scope of the embodiments. Any suitable client device can be used to access, or execute, non-steady state GFR estimate software, such as laptop computers, desktop computers, smart phones, tablet computers, gaming system, and the like.

Each computing device 800 includes one or more processors 802 that can be any suitable type of processing unit, for example a general purpose central processing unit (CPU), a reduced instruction set computer (RISC), a processor that has a pipeline or multiple processing capability including having multiple cores, a complex instruction set computer (CISC), a digital signal processor (DSP), an application specific integrated circuits (ASIC), a programmable logic devices (PLD), and a field programmable gate array (FPGA), among others. The computing resources can also include distributed computing devices, cloud computing resources, and virtual computing resources in general.

The computing device 800 also includes one or more memories 806, for example read only memory (ROM), random access memory (RAM), cache memory associated with the processor 802, or other memories such as dynamic RAM (DRAM), static ram (SRAM), programmable ROM (PROM), electrically erasable PROM (EEPROM), flash memory, a removable memory card or disk, a solid state drive, and so forth. The computing device 800 also includes storage media such as a storage device that can be configured to have multiple modules, such as magnetic disk drives, floppy drives, tape drives, hard drives, optical drives and media, magneto-optical drives and media, compact disk drives, Compact Disk Read Only Memory (CD-ROM), Compact Disk Recordable (CD-R), Compact Disk Rewriteable (CD-RW), a suitable type of Digital Versatile Disk (DVD) or BluRay disk, and so forth. Storage media such as flash drives, solid state hard drives, redundant array of individual disks (RAID), virtual drives, networked drives and other memory means including storage media on the processor 802, or memories 806 are also contemplated as storage devices. It can be appreciated that such memory can be internal or external with respect to operation of the disclosed embodiments. It can be appreciated that certain portions of the processes described herein can be performed using instructions stored on a computer-readable medium or media that direct a computer system to perform the process steps. Non-transitory computer-readable media, as used herein, comprises all computer-readable media except for transitory, propagating signals.

Network and communication interfaces 812 can be configured to transmit to, or receive data from, other computing devices 800 across a network 816. The network and communication interfaces 812 can be an Ethernet interface, a radio interface, a Universal Serial Bus (USB) interface, or any other suitable communications interface and can include receivers, transmitter, and transceivers. For purposes of clarity, a transceiver can be referred to as a receiver or a transmitter when referring to only the input or only the output functionality of the transceiver. Example communication interfaces 812 can include wired data transmission links such as Ethernet and TCP/IP. The communication interfaces 812 can include wireless protocols for interfacing with private or public networks 816. For example, the network and communication interfaces 812 and protocols can include interfaces for communicating with private wireless networks 816 such as a WiFi network, one of the IEEE 802.11x family of networks, or another suitable wireless network. The network and communication interfaces 812 can include interfaces and protocols for communicating with public wireless networks 816, using for example wireless protocols used by cellular network providers, including Code Division Multiple Access (CDMA) and Global System for Mobile Communications (GSM). A computing device 800 can use network and communication interfaces 812 to communicate with hardware modules such as a database or data store, or one or more servers or other networked computing resources. Data can be encrypted or protected from unauthorized access.

Mobile computing devices can include inertial components 808 and global positioning systems components (GPS components 810). The inertial components 808 and GPS components 810 can determine the terrestrial position of the mobile computing devices. Mobile computing devices can use the inertial components 808 and GPS components 810 in combination with radio transmissions received via the network and communication interfaces 812 to accurately determine the position of a mobile computing device. The position can be transmitted to other computing systems.

In various configurations, the computing device 800 can include a system bus 814 for interconnecting the various components of the computing device 800, or the computing device 800 can be integrated into one or more chips such as programmable logic device or application specific integrated circuit (ASIC). The system bus 814 can include a memory controller, a local bus, or a peripheral bus for supporting input and output devices 804, and communication interfaces 812. Example input and output devices 804 include keyboards, keypads, gesture or graphical input devices, motion input devices, touchscreen interfaces, one or more displays, audio units, voice recognition units, vibratory devices, computer mice, and any other suitable user interface.

The processor 802 and memory 806 can include nonvolatile memory for storing computer-readable instructions, data, data structures, program modules, code, microcode, and other software components for storing the computer-readable instructions in non-transitory computer-readable mediums in connection with the other hardware components for carrying out the methodologies described herein. Software components can include source code, compiled code, interpreted code, executable code, static code, dynamic code, encrypted code, or any other suitable type of code or computer instructions implemented using any suitable high-level, low-level, object-oriented, visual, compiled, or interpreted programming language.

Components of systems can include both software and hardware modules and can include one or more types of user interfaces or machine-to-machine interfaces. In various configurations, some or all of the user interfaces can execute on user equipment. User Equipment can generally include any computing device that has a CPU and the ability to send and receive data with the medical computing system. For example, a user interface can be an application or app designed to execute on user equipment such as a user's mobile computing device, tablet, or smartphone. Another example user interface can be software executing on the medical system that serves webpages that are delivered to user equipment and displayed on a web browser executing on a smartphone, a desktop computing device, or notebook computing device. In another example, a user interface can be a dedicated application designed to execute on user equipment. Interaction with the computing devices 800, user interfaces, and software for determining the non-steady state GFR estimates may include, without limitation, keyboard entry, writing from pen, stylus, finger, or the like, with a computer mouse, or other forms of input (voice recognition, etc.). The user interface for the software that determines non-steady state GFR estimates may be presented on a tablet, desktop, phone, board, or paper. In one embodiment, the user may interact with the software by writing with a smart pen on normal paper, modified paper, or a hard flat surface of their preference. User interaction with the software may take place in any of a variety of operational environments, such as an office setting, hospital setting, laboratory setting, pharmacy setting, mobile setting, and so forth, with one or more users interacting with the computing device 800 at a given time. Example messaging between medical systems and user equipment can include, but is not limited to, SMS, EMS, MMS, smart messaging, e-mail, pop-up notifications, push alerts, cookies, XML, HTML, webpages and the like.

In general, it will be apparent to one of ordinary skill in the art that at least some of the embodiments described herein can be implemented in many different embodiments of software, firmware, and/or hardware. The software and firmware code can be executed by a processor or any other similar computing device. The software code or specialized control hardware that can be used to implement embodiments is not limiting. For example, embodiments described herein can be implemented in computer software using any suitable computer software language type, using, for example, conventional or object-oriented techniques. Such software can be stored on any type of suitable computer-readable medium or media, such as, for example, a magnetic or optical storage medium. The operation and behavior of the embodiments can be described without specific reference to specific software code or specialized hardware components. The absence of such specific references is feasible, because it is clearly understood that artisans of ordinary skill would be able to design software and control hardware to implement the embodiments based on the present description with no more than reasonable effort and without undue experimentation.

Moreover, the processes described herein can be executed by programmable equipment, such as computers or computer systems and/or processors. Software that can cause programmable equipment to execute processes can be stored in any storage device, such as, for example, a computer system (nonvolatile) memory, an optical disk, magnetic tape, or magnetic disk. Furthermore, at least some of the processes can be programmed when the computer system is manufactured or stored on various types of computer-readable media.

It can also be appreciated that certain portions of the processes described herein can be performed using instructions stored on a computer-readable medium or media that direct a computer system to perform the process steps. A computer-readable medium can include, for example, memory devices such as diskettes, compact discs (CDs), digital versatile discs (DVDs), optical disk drives, or hard disk drives. A computer-readable medium can also include memory storage that is physical, virtual, permanent, temporary, semi-permanent, and/or semi-temporary.

A “computer,” “computer system,” “host,” “server,” or “processor” can be, for example and without limitation, a processor, microcomputer, minicomputer, server, mainframe, laptop, personal data assistant (PDA), wireless e-mail device, cellular phone, pager, processor, fax machine, scanner, or any other programmable device configured to transmit and/or receive data over a network. Computer systems and computer-based devices disclosed herein can include memory for storing certain software modules used in obtaining, processing, and communicating information. It can be appreciated that such memory can be internal or external with respect to operation of the disclosed embodiments.

In various embodiments disclosed herein, a single component can be replaced by multiple components and multiple components can be replaced by a single component to perform a given function or functions. Except where such substitution would not be operative, such substitution is within the intended scope of the embodiments. The computer systems can comprise one or more processors in communication with memory (e.g., RAM or ROM) via one or more data buses. The data buses can carry electrical signals between the processor(s) and the memory. The processor and the memory can comprise electrical circuits that conduct electrical current. Charge states of various components of the circuits, such as solid state transistors of the processor(s) and/or memory circuit(s), can change during operation of the circuits.

Some of the figures can include a flow diagram. Although such figures can include a particular logic flow, it can be appreciated that the logic flow merely provides an exemplary implementation of the general functionality. Further, the logic flow does not necessarily have to be executed in the order presented unless otherwise indicated. In addition, the logic flow can be implemented by a hardware element, a software element executed by a computer, a firmware element embedded in hardware, or any combination thereof.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art. Rather it is hereby intended the scope of the invention to be defined by the claims appended hereto.

We claim:

1. A computer-implemented method of determining a dose of a medication of a type that is filtered by the kidneys, comprising:
accessing an electronic medical record that includes one or more medical data selected from
an age of a patient,
a gender of the patient,
a weight of the patient,
an ethnicity of the patient,
a first measured creatinine level, Cr₁, at a first time, and
a second measure creatinine level, Cr₂, at a second time;
determining an estimated glomerular filtration rate of the kidneys of the patient based at least in part on the relationship
$${Cr}_{2} = \frac{A - ( A - B * {Cr}_{1} ) * ( {e}^{- B * t} )}{B} ;$$
and
determining the dose of the medicine for the patient based at least in part on the estimated glomerular filtration rate of the kidneys of the patient,
wherein t is the interval between the first time and the second time,
wherein A is a constant determined from the medical data applied to exactly one of the MDRD equation or the Cockcroft-Gault equation, and
wherein B is a constant, determined from the medical data applied to exactly one of the MDRD equation or the Cockcroft-Gault equation, that is multiplied by the estimated glomerular filtration rate over the interval.

2. The computer-implemented method of claim 1, wherein determining the dose further comprises:
determining a standard dose of medicine for the patient based on one or more medical data; and
determining an adjustment to the standard dose of medicine based at least in part on the estimated glomerular filtration rate of the kidney, and
wherein the dose is the standard dose modified by the adjustment to the standard dose.

3. The computer-implemented method of claim 1, wherein determining the dose further comprises:
determining a preferred blood concentration of the medicine for the patient; and
determining the dose of medicine needed to attain the preferred blood concentration of the medicine in the patient.

4. The computer-implemented method of claim 1, wherein determining the dose further comprises:
determining a preferred blood concentration of metabolites of the medicine for the patient; and
determining the dose of medicine to needed attain the preferred blood concentration of metabolites of the medicine in the patient.

5. The computer-implemented method of claim 1, wherein determining the dose further comprises:
determining a preferred range of blood concentrations of the medicine for the patient; and
determining the dose of medicine to attain a blood concentration within the preferred range of blood concentrations of the medicine in the patient.

6. The computer-implemented method of claim 1, wherein determining the dose further comprises:
determining one or more target blood concentrations in the patient of one or more of the medicine and a metabolite of the medicine; and
determining a schedule of doses of medicine to attain the one or more target blood concentrations.

7. The computer-implemented method of claim 1, wherein determining the dose further comprises:
determining, for the patient, one or more blood concentrations selected from the group consisting of
a preferred blood concentration of the medicine for the patient,
a preferred blood concentration of a metabolite of the medicine for the patient,
a preferred range of blood concentrations of the medicine for the patient,
a preferred range of blood concentrations of the metabolite of the medicine for the patient,
a minimum blood concentration of the medicine in the patient,
a minimum blood concentration of the metabolite of the medicine in the patient,
a maximum blood concentration of the medicine in the patient, and
a maximum blood concentration of the metabolite of the medicine in the patient; and
determining the dose of medicine to attain the one or more blood concentrations in the patient.

8. The computer-implemented method of claim 1, further comprising:
outputting information about the dose of the medicine; and
presenting the information on a display.

9. A non-transitory computer readable medium having instructions stored thereon that when executed by one or more processors causes the processors to:
access an electronic medical record that includes one or more medical data selected from
an age of a patient,
a gender of the patient,
a weight of the patient,
an ethnicity of the patient,
a first measured creatinine level, Cr₁, at a first time, and
a second measure creatinine level, Cr₂, at a second time;
determine an estimated glomerular filtration rate of a kidney of the patient based at least in part on the relationship
$${Cr}_{2} = \frac{A - ( A - B * {Cr}_{1} ) * ( {e}^{- B * t} )}{B} ;$$
determine a temporal correlation between the estimated glomerular filtration rate and the introduction of a drug into the patient; and
correlate a decrease in the estimated glomerular filtration rate with nephrotoxicity of the drug,
wherein t is the interval between the first time and the second time,
wherein A is a constant determined from the medical data applied to exactly one of the MDRD equation or the Cockcroft-Gault equation, and
wherein B is a constant, determined from the medical data applied to exactly one of the MDRD equation or the Cockcroft-Gault equation, that is multiplied by the glomerular filtration rate over the interval.

10. The non-transitory computer readable medium of claim 9, wherein the instructions further cause the one or more processors to:
present an indication of the nephrotoxicity of the drug.

11. A method, comprising:
obtaining one or more medical data selected from
an age of a patient,
a gender of the patient,
a weight of the patient,
an ethnicity of the patient,
a first measured creatinine level, Cr₁, at a first time, and
a second measure creatinine level, Cr₂, at a second time;
determining an estimated glomerular filtration rate of the kidneys of the patient based at least in part on the relationship
$${Cr}_{2} = \frac{A - ( A - B * {Cr}_{1} ) * ( {e}^{- B * t} )}{B} ;$$
and
determining, based at least in part on the estimated glomerular filtration rate of the kidneys of the patient, one or more of
an indicia of kidney function,
an indicia of the efficacy of a medical treatment, and
an administration rate for dosing intravenous fluids,
wherein t is the interval between the first time and the second time,
wherein A is a constant determined from the medical data applied to exactly one of the MDRD equation or the Cockcroft-Gault equation, and
wherein B is a constant, determined from the medical data applied to exactly one of the MDRD equation or the Cockcroft-Gault equation, that is multiplied by the estimated glomerular filtration rate over the interval.

12. The method of claim 11, further comprising:
correlating the indicia of kidney function with historical kidney transplant data to determine a measure of correlation; and
determining a measure of kidney transplant success in the patient based on the measure of correlation.

13. The method of claim 11, further comprising:
correlating the indicia of kidney function with an expected range of kidney function to determine a measure of correlation; and
quantifying a measure of decreased kidney function in the patient based on the measure of correlation.

14. The method of claim 13, further comprising:
diagnosing the patient as having an acute kidney injury based at least in part on the quantified measure of decreased kidney function.

15. The method of claim 11, wherein the medical treatment is the administration of a drug intended to improve kidney function.

16. The method of claim 11, wherein the medical treatment is a medical procedure.

17. The method of claim 16, wherein the medical procedure is a surgical medical procedure.

18. The method of claim 17, wherein the surgical medical procedure is the removal of an obstruction in a urinary tract.

19. The method of claim 11, further comprising:
dosing intravenous fluids into the patient in accordance with the determined administration rate.

20. The method of claim 11, wherein determining the estimated glomerular filtration rate of the kidneys of the patient is performed substantially continuously, wherein determining the administration rate for dosing intravenous fluids into the patient is performed substantially continuously, and further comprising:
adjusting the rate of intravenous fluids dosed into the patient substantially continuously and in accordance with the determined administration rate.
